(19) United States
(12) Patent Application Publication
(10) Pub. No.: US 2012/0009232 A1
(43) Pub. Date: Jan. 12, 2012

(54) ANTIPERSPIRANT COMPOSITIONS AND METHODS FOR MANUFACTURING ANTIPERSPIRANT COMPOSITIONS

(75) Inventors: Travis T. Yarlagadda, Phoenix, AZ (US); Thomas Doering, Scottsdale, AZ (US); Charlie Varker, Phoenix, AZ (US); Bruce R. Cox, Scottsdale, AZ (US)

(73) Assignee: The Dial Corporation, Scottsdale, AZ (US)

(21) Appl. No.: 12/832,165

(22) Filed: Jul. 8, 2010

(51) Int. Cl. A61K 8/92; A61Q 15/00; B28B 1/16; A61K 8/02; B28B 1/14

(52) U.S. Cl. 424/401; 424/65; 264/299; 264/255

(57) ABSTRACT

Antiperspirant compositions and methods for fabricating antiperspirant compositions are provided. In accordance with an exemplary embodiment, an antiperspirant composition comprises an active antiperspirant compound, stearyl alcohol, and a 14/16 chain fatty alcohol present in an amount of from about 0.5 to about 6 weight percent of the antiperspirant composition.

### FIELD OF THE INVENTION

The present invention generally relates to antiperspirant compositions and methods for manufacturing antiperspirant compositions, and more particularly relates to antiperspirant compositions with minimal or no visible surface crystal formation and methods for manufacturing such compositions.

### BACKGROUND OF THE INVENTION

Antiperspirants and deodorants are popular personal care products used to prevent or eliminate perspiration and body odor caused by perspiration. Antiperspirants typically prevent the secretion of perspiration by blocking or plugging sweat-secreting glands, such as those located at the underarms. Deodorants counteract or mask the unwanted odors caused by bacterial flora in secreted perspiration.

Antiperspirant solid sticks are desired by a large majority of the population because of the presence of active antiperspirant compounds that block or prevent the secretion of perspiration and its accompanying odors and because of their ease of application. A solid antiperspirant product is applied to the skin by swiping or rubbing the stick across the skin, typically of the underarm. Antiperspirant compositions often comprise stearyl alcohol as a desired structurant that imparts the antiperspirant stick with its hardness. Stearyl alcohol causes the antiperspirant product to maintain its hardness and solid stick form so that a user can swipe the product against the skin without the stick crumbling apart. However, while not wishing to be bound by theory, it is believed that, after manufacture, the stearyl alcohol may be responsible for visible crystals that form on the surface of the antiperspirant stick. These crystals can cause a visibly inhomogeneous and unpleasant surface.

Accordingly, it is desirable to provide antiperspirant compositions that do not exhibit surface crystals upon manufacture. In addition, it is desirable to provide methods for manufacturing antiperspirant compositions that do not exhibit surface crystals upon manufacture. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Antiperspirant compositions and methods for fabricating antiperspirant compositions are provided. In accordance with an exemplary embodiment, an antiperspirant composition comprises an active antiperspirant compound, stearyl alcohol, and a 14/16 chain fatty alcohol present in an amount of from about 0.5 to about 6 weight percent of the antiperspirant composition.

In accordance with another exemplary embodiment, an antiperspirant composition comprises an active antiperspirant compound, stearyl alcohol, and a 14/16 chain fatty alcohol present in a 14/16 chain fatty alcohol:stearyl alcohol ratio in the range of from about 1:6 to about 1:10.

In accordance with an exemplary embodiment, a method for fabricating an antiperspirant composition is provided. The method comprises combining stearyl alcohol and a 14/16 chain fatty alcohol, the 14/16 chain fatty alcohol in an amount of from about 0.5 to about 6 weight percent of the antiperspirant composition. The stearyl alcohol and the 14/16 chain fatty alcohol are melted. A melted mixture is formed. The melted mixture is poured into a mold and is allowed to cool to ambient temperature.

### BRIEF DESCRIPTION OF THE DRAWINGS

The present invention will hereinafter be described in conjunction with the following drawing figures, wherein like numerals denote like elements, and wherein:

FIG. 1 is a perspective view of an antiperspirant composition in accordance with an exemplary embodiment; and

FIG. 2 is a top view of the antiperspirant composition of FIG. 1.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The various embodiments contemplated herein relate to an antiperspirant composition that comprises stearyl alcohol as a desired structurant but that does not exhibit visible surface crystals upon manufacture. In this regard, the various embodiments comprise cetyl alcohol, myristyl alcohol, or a combination thereof. As noted above, while stearyl alcohol is a desired structurant in antiperspirant compositions because of its ability to impart structure and hardness to the compositions, in certain compositions it may cause visible crystals to form at the surface of the compositions after manufacture. It is believed that the cetyl alcohol, myristyl alcohol, or a combination thereof, present in sufficient amounts of the antiperspirant composition, forms a fatty alcohol matrix with the stearyl alcohol that prevents the visible crystals from forming.

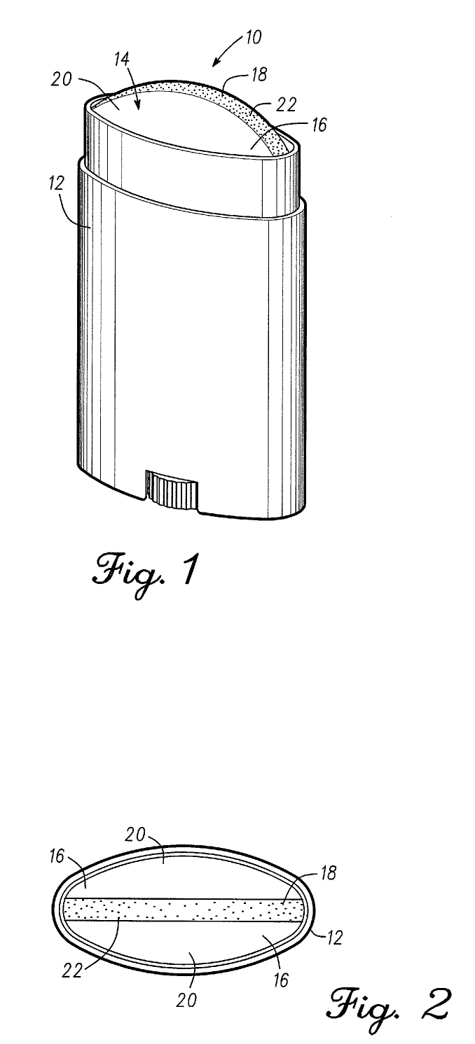

In one exemplary embodiment, the antiperspirant composition contemplated herein is a uniform solid stick. In another exemplary embodiment, referring to FIGS. 1 and 2, an antiperspirant composition 10 comprises a first portion 16 and a second portion 18. The term “portion,” as used herein, includes the section or sections of the antiperspirant composition having the same formula; for example, two sections having the same formula but separated by a third section (for example, a central stripe) having a different formula constitute a single “portion.” First portion 16 may have a color different from that of second portion 18 or the portions may be of the same color. Antiperspirant composition 10 has an application surface 14 that is substantially dome-shaped and that is configured to be applied to skin, such as, for example, an underarm. Antiperspirant composition 10 also may be disposed in a container or dispenser 12 for dispensing first portion 16 and second portion 18 to the skin.

In one exemplary embodiment, first portion 16 is an outer portion and second portion 18 is an inner portion and application surface 14 comprises a surface 20 of first portion 16 that is bisected by an adjacent surface 22 of second portion 18. First portion 16 is positioned on outside regions of the antiperspirant product 10 while second portion 18 is positioned between the regions of first portion 16. This configuration is a preferred configuration because the first portion has a melting point that is higher than the melting point of the second portion. During manufacture, as described in more detail below, the second portion is formed after the first portion and, thus, will cool before melting the already-prepared first portion. However, it will be appreciated that the invention is not limited to the configuration of first portion 16 and second portion 18 illustrated in FIGS. 1 and 2. Rather, it will be appreciated that second portion 18 can be the outer portion and first portion 16 can be the inner portion. For a thorough explanation of methods for manufacturing antiperspirant products having the form illustrated in FIGS. 1 and 2, see U.S. Pat. No. 6,723,269 B2, issued Apr. 20, 2004 and owned by the same assignee hereof, which is incorporated herein in its entirety.

It will be appreciated that first portion 16 and second portion 18 also may have other configurations. For example, second portion 18 may be completely surrounded by first portion 16 or vice versa. Alternatively, rather than forming one strip bisecting first portion 16, second portion 18 may form two or more strips. Antiperspirant product 10 may also comprise a third portion and other additional portions that do not comprise the compositions of first portion 16 and second portion 18. First portion 16 and second portion 18 may take any other configuration suitable for applying the portions to skin.

The various embodiments of the antiperspirant composition contemplated herein comprise a water-soluble active antiperspirant compound. Active antiperspirant compounds contain at least one active ingredient, typically metal salts, that are thought to reduce perspiration by diffusing through the sweat ducts of apocrine glands (sweat glands responsible for body odor) and hydrolyzing in the sweat ducts, where they combine with proteins to form an amorphous metal hydroxide agglomerate, plugging the sweat ducts so perspiration cannot diffuse to the skin surface. Some active antiperspirant compounds that may be used in the first and/or second portions include astringent metallic salts, especially inorganic and organic salts of aluminum, zirconium, and zinc, as well as mixtures thereof. Particularly preferred are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b}x(H₂O), wherein a is from 2 to about 5; the sum of a and b is about 6; x is from about 1 to about 6; and wherein a, b, and x may have non-integer values. Exemplary zirconium salts include those having the general formula ZrO(OH)₂₋ₐClₐx(H₂O), wherein a is from about 1.5 to about 1.87, x is from about 1 to about 7, and wherein a and x may both have non-integer values. Particularly preferred zirconium salts are those complexes that additionally contain aluminum and glycine, commonly known as ZAG complexes. These ZAG complexes contain aluminum chlorohydroxide and zirconyl hydroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In a preferred embodiment, the antiperspirant compound is aluminum zirconium pentachlorohydrex glycine complex or aluminum zirconium trichlorohydrex glycine complex. In another embodiment, the antiperspirant composition comprises an active antiperspirant compound present in the amount of 20 to about 25 weight percent (USP). As used herein, weight percent (USP) or wt. % (USP) of an antiperspirant salt is calculated as anhydrous weight percent in accordance with the U.S.P. method, as is known in the art. This calculation excludes any bound water and glycine.

Further included in the antiperspirant composition is stearyl alcohol. Stearyl alcohol is a fatty alcohol that serves as a structurant that provides, at least in part, the solid consistency of the antiperspirant composition. In one exemplary embodiment, stearyl alcohol is present in the antiperspirant composition in an amount of from about 16 to about 22 wt. %, preferably from about 16 to about 18 wt. %. In this regard, in one embodiment, the stearyl alcohol, and other optional structurants as discussed below, is present in an amount sufficient to impart a hardness to the antiperspirant composition in the range of from about 200 to about 350 grams-force, preferably in the range of from about 250 to about 275 grams-force, as measured by a TA.XT2i Texture Analyzer at the following settings: pre-speed—1.0 millimeters/second (mm/s); trigger force—5.0 grams (g); test speed—1.0 mm/s; retraction speed—5.0 mm/s; distance—5.0 mm; and cycles—1. The TA.XT2i is manufactured by Stable Micro Systems Ltd. of the United Kingdom.

Other structurants and/or gellants (hereinafter referred to collectively as “structurants”) that, along with stearyl alcohol, can facilitate the solid consistency of the antiperspirant composition include naturally-occurring or synthetic waxy materials or combinations thereof. Suitable structurants, including waxes and gellants, are often selected from fatty alcohols containing from 12 to 30 carbons, such as behenyl alcohol and sterols such as lanosterol. As used herein, the term “fatty” means a long chain aliphatic group, such as at least 8 or 12 linear carbons, which is frequently not branched (linear) and is typically saturated, but which can alternatively be branched and/or unsaturated. It is possible for the fatty acid to contain a hydroxyl group, as in 12-hydroxystearic acid, for example as part of a gellant combination, and to employ amido or ester derivates thereof.

Other structurants can comprise hydrocarbon waxes such as paraffin waxes, microcrystalline waxes, ceresin, squalene, and polyethylene waxes. Other suitable structurants are waxes derived or obtained from plants or animals such as hydrogenated castor oil, hydrogenated soybean oil, carnabau, spermacetti, candelilla, beeswax, modified beeswaxes, and Montan wax and individual waxy components thereof. It is also suitable herein to employ a mixture of wax structurants. Suitable mixtures of structurants can reduce the visibility of active antiperspirant compounds deposited on the skin and result in either a soft solid or a firm solid.

The various embodiments of the antiperspirant composition contemplated herein further comprise cetyl alcohol (comprising 16 carbons), myristyl alcohol (comprising 14 carbons), or a combination thereof (hereinafter, referred to collectively as a “14/16 chain fatty alcohol”). As noted above, based on information and belief, the 14/16 chain fatty alcohols likely form a fatty alcohol matrix with stearyl alcohol that prevents the formation of crystals on the surface of the antiperspirant composition upon manufacture thereof. In one exemplary embodiment, the 14/16 chain fatty alcohols are present in an amount of from about 0.5 to about 6 wt. %, preferably from about 2 to about 3 wt. %, of the antiperspirant composition.

In another exemplary embodiment, the 14/16 chain fatty alcohols and the stearyl alcohol are present in a 14/16 chain fatty alcohol:stearyl alcohol ratio of from about 1:6 to about 1:10, preferably 1:8.

In some embodiments, because of the low melting point of the 14/16 chain fatty alcohols (about 49° C. for cetyl alcohol and about 37-39° C. for myristyl alcohol) compared to stearyl alcohol (about 60° C.), the addition of the 14/16 chain fatty alcohols may cause the antiperspirant composition to have a lower than desired melting point. While it is desirable for the antiperspirant composition to slightly melt upon application to skin, it is also desirable for the antiperspirant composition to be thermally stable and maintain a solid form at 45° C. Accordingly, in an exemplary embodiment, it may be desirable to include hydrogenated castor oil in the antiperspirant composition in an amount in the range of from about 2.8 to about 7.0 wt. %, preferably from about 4 to about 6 wt. % of the antiperspirant compound. In another optional embodiment, it may be desirable to add high molecular weight (high MW) polyethylene to the antiperspirant compound. As used herein, the term “high molecular weight polyethylene” or “high MW polyethylene” means polyethylene having a molecular weight of from about 200 to about 5000 daltons (Da). High MW polyethylene has a melting point of about 70° C.-100° C. and can raise the melting point of the antiperspirant composition. In one embodiment, the antiperspirant composition comprises from about 0 to about 2 wt. % high MW polyethylene. In a preferred embodiment, the high MW polyethylene has a molecular weight of about 500 Da.

The antiperspirant compositions also may comprise a high refractive index (R.I.) hydrophobic compound. As used herein, the term “high refractive index” means a refractive index of no less than about 1.4. The high R.I. hydrophobic compound also facilitates the minimization and/or prevention of a white residue on the skin by masking the active antiperspirant salt that stays upon the skin upon evaporation of the carrier, discussed in more detail below. Examples of high R.I. hydrophobic compounds for use in the antiperspirant products include PPG-14 butyl ether, C₁₂-C₁₅ alkyl benzoate, such as Finsolv TN® available from Innospec of the United Kingdom, and phenyl dimethicone. In a preferred embodiment, the antiperspirant product comprises PPG-14 butyl ether.

In another exemplary embodiment, the antiperspirant product comprises one or more suspending agents that facilitate suspension of the active antiperspirant compound in the antiperspirant product, thereby minimizing the amount of active antiperspirant compound that settles out of the antiperspirant product during manufacture. Suitable suspending agents include clays and silicas. Examples of suitable silicas include fumed silicas and silica derivatives, such as silica dimethyl silylate. Suitable clays include bentonites, hectorites and colloidal magnesium aluminum silicates. In one exemplary embodiment, the antiperspirant product comprises about 0.1 to about 2.5 wt. % suspending agents. In another exemplary embodiment, the antiperspirant product comprises a mixture of silica and silica dimethyl silylate. In another exemplary embodiment, the antiperspirant product does not use suspending agents, but comprises high melting point waxes to prevent settling of the active ingredients. Examples of suitable high melting point waxes include hydrogenated castor oils and polyethylenes having various melting points above 65° C.

In addition to the ingredients identified above, the antiperspirant composition may comprise additives, such as those used in conventional antiperspirants. These additives include, but are not limited to, fragrances, including encapsulated fragrances, dyes, pigments, preservatives, antioxidants, moisturizers, and the like. These optional ingredients can be included in the antiperspirant composition in an amount of 0 to about 20 wt. %. In a preferred embodiment, the antiperspirant composition comprises from about 1.7 to about 2 wt. % myristyl myristate, which provides a conditioning effect to the skin.

The antiperspirant composition further comprises at least one hydrophobic carrier. An example of suitable hydrophobic carriers includes liquid siloxanes and particularly volatile polyorganosiloxanes, that is, liquid materials having a measurable vapor pressure at ambient conditions. The polyorganosiloxanes can be linear or cyclic or mixtures thereof. The linear volatile silicones generally have viscosities of less than about 5 centistokes at 25° C., while the cyclic volatile silicones have viscosities under 10 centistokes. Preferred siloxanes include cyclomethicones, which have from about 3 to about 6 silicon atoms, such as cyclotetrasiloxane, cyclopentasiloxane, and cyclohexasiloxane, and mixtures thereof. The carrier also may comprise, additionally or alternatively, nonvolatile silicones such as dimethicone and dimethicone copolyols, which have from about 2 to about 9 silicon atoms. Examples of suitable dimethicone and dimethicone copolyols include polyalkyl siloxanes, polyalkylaryl siloxanes, and polyether siloxane copolymers.

The antiperspirant compositions, according to various embodiments, can be prepared by first combining the stearyl alcohol, the 14/16 fatty alcohol, and the polyethylene, if used, and then melting them or, alternatively, melting each component and then mixing them, to form a melted mixture. The remaining ingredients can be added to the melted mixture, either separately or as one or more premixtures, to form a liquid active mixture. The active mixture is then poured into molds and permitted to cool at room temperature to form a solid stick antiperspirant product.

The following are exemplary embodiments of an antiperspirant composition, with each of the components set forth in weight percent of the antiperspirant composition. The examples are provided for illustration purposes only and are not meant to limit the various embodiments of the antiperspirant composition in any way. All materials are set forth in weight percent.

### Example 1

| Ingredient | Wt. % |
|---|---|
| Cyclopentasiloxane | 37.02 |
| Aluminum zirconium | 21.84 |
| pentachlorohydrex GLY | |
| Silica | 0.35 |
| Silica dimethy silylate | 1.38 |
| Hydrogenated castor oil | 2.84 |
| Myristyl myristate | 1.92 |
| Cetyl Alcohol | 2.50 |
| Performalene 500 | 0.75 |
| PPG-14 butyl ether | 9.80 |
| Stearyl alcohol | 20.00 |
| Fragrance | 1.60 |
| Total | 100.00, |

where Performalene 500 is polyethylene having a molecular weight of 500 Da.

The antiperspirant composition of Example 1 was manufactured by adding 712.42 grams (g) cyclopentasiloxane to a vessel and initiating agitation. With agitation continuing, 7.12 g silica and 28.6 g silica dimethyl silylate were added incrementally until all the silica was wetted. Utilizing high shear mixing, 451.87 g aluminum zirconium pentachlorohydrex GLY complex was incrementally added to form an active premix having a consistent fluid appearance without any particulates. In another mixing container, 42.60 g hydrogenated castor oil, 300 g stearyl alcohol, 147 g PPG-14 butyl ether, 28.8 g myristyl myristate, 37.5 g cetyl alcohol, and 11.25 g Performalene 500 were added and heat was initiated to melt the components. Agitation was slowly initiated as the mixture became molten. The mixture was not permitted to exceed 85° C. When all of the components were molten, with continuous agitation, 870 g of the active premix was incrementally added while the batch temperature was maintained between 64 and 69° C. Cyclopentasiloxane in the amount of 38.85 g was added while the mixture was maintained at a batch temperature of about 60° C. and mixing was continued until the mixture was homogeneous. Fragrance in the amount of 24 g was added at 60° C. The antiperspirant composition then was cooled to 53° C., poured into molds and permitted to cool to ambient temperatures. As used herein, “permitted to cool to ambient temperatures” means either that the antiperspirant composition is exposed to the ambient for a sufficient amount of time that it cools to the ambient temperature or that it is subjected to an artificial cooling means, such as a fan, refrigerator, or the like, that cools the antiperspirant composition to ambient temperatures.

### Example 2

| Ingredient | Wt. % |
|---|---|
| Cyclopentasiloxane | 33.43 |
| Aluminum zirconium | 22.20 |
| trichlorohydrex GLY | |
| Silica | 0.16 |
| Silica dimethy silylate | 0.65 |
| Hydrogenated castor oil | 4.84 |
| Myristyl myristate | 1.92 |
| Cetyl alcohol | 2.20 |
| Performalene 500 | 0.75 |
| PPG-14 butyl ether | 14.00 |
| Stearyl alcohol | 16.93 |
| Zea mays | 0.27 |
| Dye | 0.02 |
| Fragrance and zea mays | 2.63 |
| Total | 100.00 |

The antiperspirant composition of Example 2 was manufactured by adding 435.60 g cyclopentasiloxane to a vessel and initiating agitation. With agitation continuing, 2.57 g silica and 10.24 g silica dimethyl silylate were added incrementally until all the silica was wetted. Utilizing high shear mixing, 351.59 g aluminum zirconium trichlorohydrex GLY complex was incrementally added to form an active premix having a consistent fluid appearance without any particulates.

Next, an outer phase of an antiperspirant composition similar to that illustrated in FIGS. 1 and 2 was formulated. In another mixing container, 33.88 g hydrogenated castor oil, 116.90 g stearyl alcohol, 98.0 g PPG-14 butyl ether, 13.4 g myristyl myristate, 15.4 g cetyl alcohol, and 5.3 g Performalene 500 were added and heat was initiated to melt the components. Agitation was slowly added as the mixture became molten. The mixture was not permitted to exceed 99° C. (Another option is to melt the polyethylene separately and pour it into the molten base.) Once the mixture had become thoroughly molten, it was allowed to cool to 70° C. With continuous agitation, 376.74 g of the active premix was incrementally added while the batch temperature was maintained at between 64-69° C. Mixing was continued until the mixture was homogenous. Cyclopentasiloxane in the amount of 40.4 g was added while the mixture was maintained at a batch temperature of about 60° C. and mixing was continued until the mixture was homogeneous. The outer phase of the antiperspirant composition then was cooled to about 51.5° C., poured into a suitable mold with an insert in place and the insert was pulled when the molten outer phase had reached the temperature of 35° C., forming a cavity therein.

The inner phase of antiperspirant composition then was formulated. In yet another mixing container, 31.46 g hydrogenated castor oil, 113.1 g stearyl alcohol, 91 g PPG-14 butyl ether, 12.48 g myristyl myristate, 14.3 g cetyl alcohol, and 4.88 g Performalene 500 were added and heat was initiated to melt the components. Agitation was slowly added as the mixture became molten. The mixture was not permitted to exceed 99° C. (Another option is to melt the polyethylene separately and pour it into the molten base.) Once the mixture had become thoroughly molten, it was allowed to cool to 70° C. With continuous agitation, 284.25 g of the active premix was incrementally added while the batch temperature was maintained at between 64-69° C. Mixing was continued until the mixture was homogenous. Then 17.81 g of fragrance encapsulated in zea mays, cyclopentasiloxane in the amount of 49.08 g, and dye in the amount of 0.45 g was added while the mixture was maintained at a batch temperature of about 60° C. and mixing was continued until the mixture was homogeneous. Fragrance in the amount of 31.2 g was added and the inner phase of the antiperspirant composition then was cooled to about 51.5° C. The inner phase was poured into the cavity within the outer phase formed by the removed insert. The antiperspirant composition then was permitted to cool to room temperature.

Accordingly, various embodiments relating to an antiperspirant composition that comprises stearyl alcohol as a desired structurant but that does not exhibit surface crystals upon manufacture have been provided. In this regard, the various embodiments comprise cetyl alcohol, myristyl alcohol, or a combination thereof. As noted above, while stearyl alcohol is a desired structurant in antiperspirant compositions because of its ability to impart structure and hardness to the compositions, in certain compositions it may cause crystals to form at the surface of the compositions after manufacture. It is believed that the cetyl alcohol, myristyl alcohol or a combination thereof, present in sufficient amounts in the antiperspirant composition, forms a fatty alcohol matrix with the stearyl alcohol that prevents the crystals from forming.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant composition comprising:
an active antiperspirant compound;
stearyl alcohol; and
a 14/16 chain fatty alcohol present in an amount of from about 0.5 to about 6 weight percent of the antiperspirant composition.

2. The antiperspirant composition of claim 1, wherein the 14/16 chain fatty alcohol is cetyl alcohol.

3. The antiperspirant composition of claim 1, wherein the 14/16 chain fatty alcohol is myristyl alcohol.

4. The antiperspirant composition of claim 1, wherein the 14/16 chain fatty alcohol is a combination of cetyl alcohol and myristyl alcohol.

5. The antiperspirant composition of claim 1, wherein the 14/16 chain fatty alcohol is present in an amount of from about 2 to about 3 weight percent of the antiperspirant composition.

6. The antiperspirant composition of claim 1, wherein the 14/16 chain fatty alcohol is present in a 14/16 chain fatty alcohol:stearyl alcohol ratio of from about 1:6 to about 1:10.

7. The antiperspirant composition of claim 1, wherein the 14/16 chain fatty alcohol is present in a 14/16 chain fatty alcohol:stearyl alcohol ratio of about 1:8.

8. The antiperspirant composition of claim 1, further comprising a high molecular weight polyethylene.

9. The antiperspirant composition of claim 1, further comprising hydrogenated castor oil in an amount of from about 2.8 to about 7.0 weight percent.

10. The antiperspirant composition of claim 1, further comprising hydrogenated castor oil in an amount of from about 4 to about 6 weight percent.

11. The antiperspirant composition of claim 1, wherein the antiperspirant composition is a uniform solid stick.

12. The antiperspirant composition of claim 1, wherein the antiperspirant composition comprises an outer phase and an inner phase that bisects the outer phase.

13. An antiperspirant composition comprising:
an active antiperspirant compound;
stearyl alcohol; and
a 14/16 chain fatty alcohol present in a 14/16 chain fatty alcohol:stearyl alcohol ratio in the range of from about 1:6 to about 1:10.

14. The antiperspirant composition of claim 13, wherein the 14/16 chain fatty alcohol comprises one selected from the group consisting of cetyl alcohol, myristyl alcohol, and a combination thereof.

15. The antiperspirant composition of claim 13, wherein the 14/16 chain fatty alcohol is present in a 14/16 chain fatty alcohol:stearyl alcohol ratio of about 1:8.

16. The antiperspirant composition of claim 13, wherein the 14/16 chain fatty alcohol is present in an amount of from about 2 to about 3 weight percent of the antiperspirant composition.

17. The antiperspirant composition of claim 13, further comprising a high molecular weight polyethylene.

18. The antiperspirant composition of claim 13, further comprising hydrogenated castor oil in an amount of from about 2.8 to about 7.0 weight percent.

19. The antiperspirant composition of claim 18, further comprising the hydrogenated castor oil in an amount of from about 4 to about 6 weight percent.

20. The antiperspirant composition of claim 13, wherein the antiperspirant composition is a uniform solid stick.

21. The antiperspirant composition of claim 13, wherein the antiperspirant composition comprises an outer phase and an inner phase that bisects the outer phase.

22. A method for fabricating an antiperspirant composition, the method comprising the steps of:
combining stearyl alcohol and a 14/16 chain fatty alcohol, the 14/16 chain fatty alcohol in an amount of from about 0.5 to about 6 weight percent of the antiperspirant composition;
melting the stearyl alcohol and the 14/16 chain fatty alcohol;
forming a melted mixture;
pouring the melted mixture into a mold; and
allowing the melted mixture to cool to ambient temperature.

23. The method of claim 22, wherein the step of melting the stearyl alcohol and the 14/15 chain fatty alcohol is performed before the step of combining.

24. The method of claim 22, wherein the step of pouring comprises pouring the melted mixture into the mold having inserts and further comprising the steps of:
removing the inserts after the melted mixture has cooled to a first temperature and forming a cavity therein; and
pouring an inner phase mixture into the cavity.

25. The method of claim 22, wherein the step of combining comprises combining stearyl alcohol and a 14/16 chain fatty alcohol comprising one selected from the group consisting of cetyl alcohol, myristyl alcohol and a combination thereof.

26. The method of claim 22, wherein the step of combining comprises combining stearyl alcohol and a 14/16 chain fatty alcohol, the 14/16 chain fatty alcohol in an amount of from about 2 to about 3 weight percent of the antiperspirant composition.

27. The method of claim 22, wherein the step of combining comprises combining stearyl alcohol and a 14/16 chain fatty alcohol, the stearyl alcohol and the 14/16 chain fatty alcohol present in a 14/16 chain fatty alcohol:stearyl alcohol ratio in the range of from about 1:6 to about 1:10.

28. The method of claim 27, wherein the step of combining comprises combining stearyl alcohol and a 14/16 chain fatty alcohol, the stearyl alcohol and the 14/16 chain fatty alcohol present in a 14/16 chain fatty alcohol:stearyl alcohol ratio in the range of about 1:8.

29. The method of claim 22, further comprising the step of adding an active antiperspirant compound to the melted mixture before the step of pouring.

30. The method of claim 22, further comprising the step of combining hydrogenated castor oil in the amount of from about 2.8 to about 7.0 weight percent with the stearyl alcohol and the 14/16 chain fatty alcohol, wherein the step of combining the hydrogenated castor oil is performed before or during the step of forming the melted mixture.

31. The method of claim 30, further comprising the step of combining the hydrogenated castor oil in the amount of from about 4 to about 6 weight percent with the stearyl alcohol and the 14/16 chain fatty alcohol, wherein the step of combining the hydrogenated castor oil is performed before or during the step of forming the melted mixture.

32. The method of claim 22, further comprising the step of combining high molecular weight polyethylene with the stearyl alcohol and the 14/16 chain fatty alcohol, wherein the step of combining the high molecular weight polyethylene is performed before or during the step of forming the melted mixture.
